(19) United States
(12) Patent Application Publication
(10) Pub. No.: US 2010/0312013 A1
(43) Pub. Date: Dec. 9, 2010

(54) STEROSELECTIVE SYNTHESIS OF CERTAIN TRIFLUOROMETHYL-SUBSTITUTED ALCOHOLS

(75) Inventors: Daniel R. FANDRICK, Danbury, CT (US); Nathan K. YEE, Danbury, CT (US); Jinhua J. SONG, Hopewell Junction, NY (US); Jonathan T. REEVES, New Milford, CT (US)

Correspondence Address: MICHAEL P. MORRIS;BOEHRINGER INGELHEIM USA CORPORATION, 900 RIDGEBURY ROAD, P O BOX 368, RIDGEFIELD, CT, 06877-0368, US

(73) Assignee: BOEHRINGER INGELHEIM INTERNATIONAL GMBH, Ingelheim am Rhein (DE)

(21) Appl. No.: 12/788,557

(22) Filed: May 27, 2010

Related U.S. Application Data
(60) Provisional application No. 61/183,607, filed on Jun. 3, 2009.

(51) Int. Cl. C07C 233/75

(52) U.S. Cl. 564/169

(57) ABSTRACT

A process for synthesis of a compound of Formula (X)

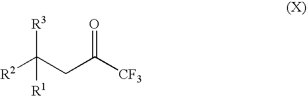

wherein:
R¹ is an aryl group substituted with one to three substituent groups,
   wherein each substituent group of R¹ is independently C₁-C₅ alkyl, aminocarbonyl, alkylaminocarbonyl, dialkylaminocarbonyl, halogen, carboxy, cyano, or trifluoromethyl,
      wherein each substituent group of R¹ is optionally independently substituted with one to three substituents selected from C₁-C₃ alkyl, C₁-C₃ alkoxy, phenyl, and alkoxyphenyl; and
R² and R³ are each independently C₁-C₅ alkyl.

### FIELD OF THE INVENTION

The present invention relates to the stereoselective synthesis of certain trifluoromethyl-substituted alcohols.

### BACKGROUND OF THE INVENTION

Trifluoromethyl-substituted alcohols of formula (I) have been described as ligands that bind to the glucocorticoid receptor. These compounds are potential therapeutics in treating a number of diseases modulated by glucocorticoid receptor function, including inflammatory, autoimmune and allergic disorders. Examples of these compounds are described in U.S. Pat. Nos. 7,268,152; 7,189,758; 7,186,864; 7,074,806; 6,960,581; 6,903,215; and 6,858,627, which are each incorporated herein by reference in their entireties and are hereinafter termed “the Trifluoromethyl-Substituted Alcohol Patent Applications”.

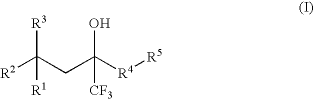

It is well known in the art that enantiomers of a particular compound can have different biological properties including efficacy, toxicity, and pharmacokinetic properties. Thus, it is often desirable to administer one enantiomer of a racemic therapeutic compound.

The synthetic methods disclosed in the patent applications cited above describe the synthesis of racemic products. Separation of enantiomers was accomplished by chiral HPLC and may be accomplished by other conventional ways of separating enantiomers. Chiral HPLC and other enantiomer separation method, however, are generally unsuitable for large-scale preparation of a single enantiomer. Thus, a stereoselective synthesis for preparation of these compounds would be highly desirable.

The present invention discloses a synthesis of certain compounds of Formula (X)

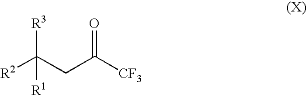

which are key intermediates in the synthesis of enantiomerically pure compounds of Formula (I).

### SUMMARY OF THE INVENTION

The instant invention is directed to a process for synthesis of a compound of Formula (X)

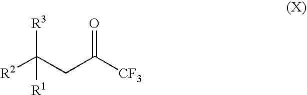

wherein:
R¹ is an aryl group substituted with one to three substituent groups,
wherein each substituent group of R¹ is independently C₁-C₅ alkyl, aminocarbonyl, alkylaminocarbonyl, dialkylaminocarbonyl, halogen, carboxy, cyano, or trifluoromethyl,
   wherein each substituent group of R¹ is optionally independently substituted with one to three substituents selected from C₁-C₃ alkyl, C₁-C₃ alkoxy, phenyl, and alkoxyphenyl; and
      R² and R³ are each independently C₁-C₅ alkyl,
      the process comprising:
(a) reacting Meldrum's acid of Formula (A) with a carbonyl compound of Formula (B) bearing R² and R³, in a suitable solvent, in the presence of a suitable base, to provide the alkylidene-dione of Formula (C)

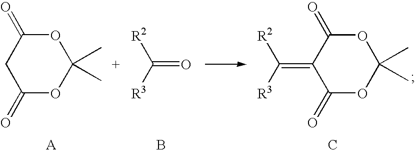

(b) reacting the alkylidene-dione of Formula (C) with a suitable organohalide of Formula (D) wherein Hal is Br or I, in the presence of a suitable organometallic reagent such as an alkyl magnesium chloride, in a suitable solvent, and subsequently adding water and a suitable acid, such as hydrochloric acid, to the reaction mixture and heating to form the acid of Formula (E)

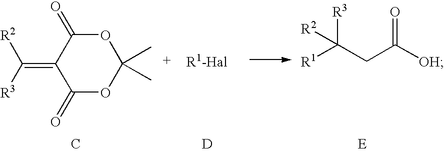

and
(c) reacting the acid of Formula (E) with a trifluoromethyl reagent such as trifluoroacetic anhydride, in a suitable solvent, in the presence of a suitable base, to provide a compound of Formula (X)

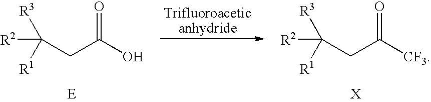

Another aspect of the invention includes a process for the synthesis of a compound of Formula (X), wherein:

R¹ is an aryl group substituted with one to three substituent groups,
wherein each substituent group of R¹ is independently C₁-C₅ alkyl, aminocarbonyl, alkylaminocarbonyl, halogen, carboxy, cyano, or trifluoromethyl,
   wherein each substituent group of R¹ is optionally independently substituted with one to three substituents selected from C₁-C₃ alkyl, phenyl, and alkoxyphenyl; and
      R² and R³ are each independently C₁-C₃ alkyl,
      the process as set forth above with R¹, R², and R³ as specified.

In an aspect of the invention, the suitable solvent of step (a) is diethyl ether, dipropyl ether, diisopropyl ether, dibutyl ether, tetrahydrofuran (THF), ethylene glycol dimethyl ether (DME), tert-butyl methyl ether (MTBE), or a mixture thereof, preferably diethyl ether or THF.

In an aspect of the invention, the suitable base for step (a) is pyridine, piperidine, pyrrolidine, ammonia, or morpholine, preferably piperidine.

In an aspect of the invention, the carbonyl compound (B) for step (a) is acetone, cyclohexanone, 2-butanone, 3-pentanone, cyclopentanone, or any other cyclic or acyclic dialkyl ketone.

In an aspect of the invention, the suitable solvent of step (b) is diethyl ether, dipropyl ether, diisopropyl ether, dibutyl ether, THF, DME, MTBE, toluene, or a mixture thereof, preferably diethyl ether or THF.

In an aspect of the invention, the suitable organometallic reagent of step (b) is isopropylmagnesium chloride, cyclopentylmagnesium chloride, n-butylmagnesium chloride, or tert-butylmagnesium chloride, preferably isopropylmagnesium chloride.

In an aspect of the invention, the suitable solvent of step (c) is dipropyl ether, diisopropyl ether, dibutyl ether, MTBE, toluene, dichloromethane, or a mixture thereof, preferably toluene.

In another aspect of the invention, the suitable base of step (c) is pyridine, 2-chloropyridine, 2,6-lutidine, or 2,4,6-collidine, preferably pyridine.

In still another aspect of the invention, the suitable trifluoromethyl reagent of step (c) is trifluoroacetic anhydride or trifluoroacetyl chloride, preferably trifluoroacetic anhydride.

When R¹ is an optionally substituted bromophenyl group, then reaction of a compound of Formula (X) with carbon dioxide, in a suitable solvent, in the presence of a suitable base and a suitable organometallic reagent, provides a compound of Formula (X′)

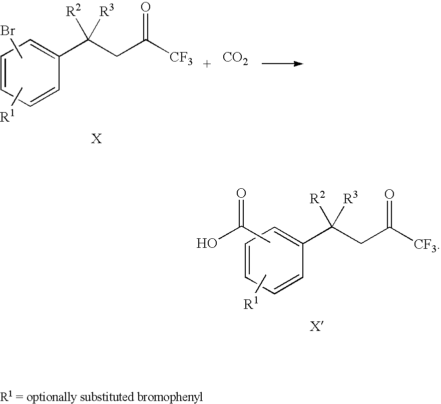

In other aspects of the invention, the process of making the compound of Formula (X′), the suitable solvent is THF, 2-methyltetrahydrofuran, MTBE, 1,2-dimethoxyethane, or toluene, or a mixture thereof, preferably THF; the suitable base is sodium hydride, lithium hydride, or calcium hydride, preferably sodium hydride; and the suitable organometallic reagent is isopropylmagnesium chloride, isopropylmagnesium chloride-lithium chloride, n-butylmagnesium chloride, di-n-butylmagnesium, cyclohexylmagnesium chloride, cyclopentylmagnesium chloride, or any other secondary alkylmagnesium chloride, preferably isopropylmagnesium chloride-lithium chloride.

The reaction of a compound of Formula (X′) with a suitable amine, in the presence of suitable reagent, in the presence of a suitable base, in a suitable solvent, provides a compound of Formula (X″)

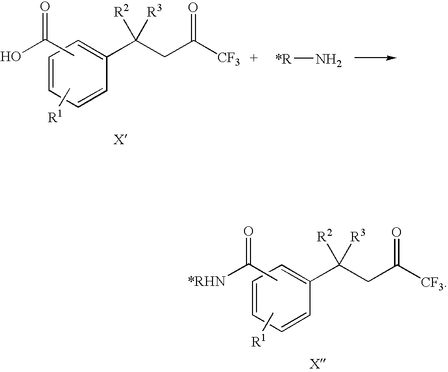

In other aspects of the invention, the process of making the compound of Formula (X″), the suitable solvent is THF, 2-methyltetrahydrofuran, MTBE, 1,2-dimethoxyethane, or toluene, or a mixture thereof; the suitable amine is any chiral 1-phenylethylamine or any chiral 1-alkyl-1-arylamine; the suitable reagent is thionyl chloride, oxalyl chloride, 1,1′-carbonyldiimidazole, trimethylacetyl chloride, or isobutylchloroformate, preferably thionyl chloride; the suitable base is 2,6-lutidine, pyridine, 2,4,6-collidine, or ethyldiisopropylamine, preferably 2,6-lutidine.

The compound of Formula (X), (X′), or (X″) may be converted to another compound of Formula (X), (X′), or (X″) by reactions known to one skilled in the art.

It should be noted that the invention should be understood to include none, some, or all of these various aspects in various combination.

### DETAILED DESCRIPTION OF THE INVENTION

### Definition of Terms and Conventions Used

Terms not specifically defined herein should be given the meanings that would be given to them by one of skill in the art in light of the disclosure and the context. As used in the specification and appended claims, however, unless specified to the contrary, the following terms have the meaning indicated and the following conventions are adhered to.

### A. Chemical Nomenclature, Terms, and Conventions

In the groups, radicals, or moieties defined below, the number of carbon atoms is often specified preceding the group, for example, C₁-C₁₀ alkyl means an alkyl group or radical having 1 to 10 carbon atoms. The term “lower” applied to any carbon-containing group means a group containing from 1 to 8 carbon atoms, as appropriate to the group (i.e., a cyclic group must have at least 3 atoms to constitute a ring). In general, for groups comprising two or more subgroups, the last named group is the radical attachment point, for example, “alkylaryl” means a monovalent radical of the formula Alk-Ar-, while “arylalkyl” means a monovalent radical of the formula Ar-Alk- (where Alk is an alkyl group and Ar is an aryl group). Furthermore, the use of a term designating a monovalent radical where a divalent radical is appropriate shall be construed to designate the respective divalent radical and vice versa. Unless otherwise specified, conventional definitions of terms control and conventional stable atom valences are presumed and achieved in all formulas and groups.

The terms “alkyl” or “alkyl group” mean a branched or straight-chain saturated aliphatic hydrocarbon monovalent radical. This term is exemplified by groups such as methyl, ethyl, n-propyl, 1-methylethyl (isopropyl), n-butyl, n-pentyl, 1,1-dimethylethyl (tert-butyl), and the like. It may be abbreviated “Alk”.

The terms “alkenyl” or “alkenyl group” mean a branched or straight-chain aliphatic hydrocarbon monovalent radical containing at least one carbon-carbon double bond. This term is exemplified by groups such as ethenyl, propenyl, n-butenyl, isobutenyl, 3-methylbut-2-enyl, n-pentenyl, heptenyl, octenyl, decenyl, and the like.

The terms “alkynyl” or “alkynyl group” mean a branched or straight-chain aliphatic hydrocarbon monovalent radical containing at least one carbon-carbon triple bond. This term is exemplified by groups such as ethynyl, propynyl, n-butynyl, 2-butynyl, 3-methylbutynyl, n-pentynyl, heptynyl, octynyl, decynyl, and the like.

The terms “alkylene” or “alkylene group” mean a branched or straight-chain saturated aliphatic hydrocarbon divalent radical having the specified number of carbon atoms. This term is exemplified by groups such as methylene, ethylene, propylene, n-butylene, and the like, and may alternatively and equivalently be denoted herein as -(alkyl)-.

The terms “alkenylene” or “alkenylene group” mean a branched or straight-chain aliphatic hydrocarbon divalent radical having the specified number of carbon atoms and at least one carbon-carbon double bond. This term is exemplified by groups such as ethenylene, propenylene, n-butenylene, and the like, and may alternatively and equivalently be denoted herein as -(alkylenyl)-.

The terms “alkynylene” or “alkynylene group” mean a branched or straight-chain aliphatic hydrocarbon divalent radical containing at least one carbon-carbon triple bond. This term is exemplified by groups such as ethynylene, propynylene, n-butynylene, 2-butynylene, 3-methylbutynylene, n-pentynylene, heptynylene, octynylene, decynylene, and the like, and may alternatively and equivalently be denoted herein as -(alkynyl)-.

The terms “alkoxy” or “alkoxy group” mean a monovalent radical of the formula AlkO—, where Alk is an alkyl group. This term is exemplified by groups such as methoxy, ethoxy, propoxy, isopropoxy, butoxy, sec-butoxy, tert-butoxy, pentoxy, and the like.

The terms “alkoxycarbonyl” or “alkoxycarbonyl group” mean a monovalent radical of the formula AlkO-C(O)—, where Alk is alkyl. Exemplary alkoxycarbonyl groups include methoxycarbonyl, ethoxycarbonyl, tert-butyloxycarbonyl, and the like.

The term “alkoxycarbonylamino” or “alkoxycarbonylamino group” mean a monovalent radical of the formula ROC(O)NH—, where R is lower alkyl.

The terms “alkylcarbonylamino” or “alkylcarbonylamino group” or “alkanoylamino” or “alkanoylamino groups” mean a monovalent radical of the formula AlkC(O)NH—, where Alk is alkyl. Exemplary alkylcarbonylamino groups include acetamido (CH₃C(O)NH—).

The terms “alkylaminocarbonyloxy” or “alkylaminocarbonyloxy group” mean a monovalent radical of the formula AlkNHC(O)O—, where Alk is alkyl.

The terms “amino” or “amino group” mean an —NH₂ group.

The terms “alkylamino” or “alkylamino group” mean a monovalent radical of the formula (Alk)NH—, where Alk is alkyl. Exemplary alkylamino groups include methylamino, ethylamino, propylamino, butylamino, tert-butylamino, and the like.

The terms “dialkylamino” or “dialkylamino group” mean a monovalent radical of the formula (Alk)(Alk)N—, where each Alk is independently alkyl. Exemplary dialkylamino groups include dimethylamino, methylethylamino, diethylamino, dipropylamino, ethylpropylamino, and the like.

The terms “aminocarbonyl”, “alkylaminocarbonyl” or “dialkylaminocarbonyl” mean a monovalent radical of the formula R₂NC(O)—, where the R is independently hydrogen or alkyl.

The terms “substituted amino” or “substituted amino group” mean a monovalent radical of the formula —NR₂, where each R is independently a substituent selected from hydrogen or the specified substituents (but where both Rs cannot be hydrogen). Exemplary substituents include alkyl, alkanoyl, aryl, arylalkyl, cycloalkyl, heterocyclyl, heteroaryl, heteroarylalkyl, and the like.

The terms “alkoxycarbonylamino” or “alkoxycarbonylamino group” mean a monovalent radical of the formula AlkOC(O)NH—, where Alk is alkyl.

The terms “halogen” or “halogen group” mean a fluoro, chloro, bromo, or iodo group.

The term “halo” means one or more hydrogen atoms of the group are replaced by halogen groups.

The terms “alkylthio” or “alkylthio group” mean a monovalent radical of the formula AlkS—, where Alk is alkyl. Exemplary groups include methylthio, ethylthio, n-propylthio, isopropylthio, n-butylthio, and the like.

The terms “sulfonyl” or “sulfonyl group” mean a divalent radical of the formula —SO₂—.

The terms “aminosulfonyl”, “alkylaminosulfonyl” and “dialkylaminosulfonyl” mean a monovalent radical of the formula R₂N—SO₂—, wherein R is independently hydrogen or alkyl

The terms “aryl” or “aryl group” mean an aromatic carbocyclic monovalent or divalent radical of from 6 to 14 carbon atoms having a single ring (e.g., phenyl or phenylene) or multiple condensed rings (e.g., naphthyl or anthranyl). Unless otherwise specified, the aryl ring may be attached at any suitable carbon atom which results in a stable structure and, if substituted, may be substituted at any suitable carbon atom which results in a stable structure. Exemplary aryl groups include phenyl, naphthyl, anthryl, phenanthryl, indanyl, indenyl, biphenyl, and the like. It may be abbreviated “Ar”.

The term “compounds of the invention” and equivalent expressions are meant to embrace compounds of Formula (I) as herein described, including the tautomers, the prodrugs, the salts, particularly the pharmaceutically acceptable salts, and the solvates and hydrates thereof, where the context so permits. In general and preferably, the compounds of the invention and the formulas designating the compounds of the invention are understood to only include the stable compounds thereof and exclude unstable compounds, even if an unstable compound might be considered to be literally embraced by the compound formula. Similarly, reference to intermediates, whether or not they themselves are claimed, is meant to embrace their salts and solvates, where the context so permits. For the sake of clarity, particular instances when the context so permits are sometimes indicated in the text, but these instances are purely illustrative and it is not intended to exclude other instances when the context so permits.

The terms “optional” or “optionally” mean that the subsequently described event or circumstances may or may not occur, and that the description includes instances where the event or circumstance occurs and instances in which it does not. For example, “optionally substituted aryl” means that the aryl radical may or may not be substituted and that the description includes both substituted aryl radicals and aryl radicals having no substitution.

The terms “stable compound” or “stable structure” mean a compound that is sufficiently robust to survive isolation to a useful degree of purity from a reaction mixture, and formulation into an efficacious therapeutic or diagnostic agent. For example, a compound which would have a “dangling valency” or is a carbanion is not a compound contemplated by the invention.

The term “substituted” means that any one or more hydrogens on an atom of a group or moiety, whether specifically designated or not, is replaced with a selection from the indicated group of substituents, provided that the atom's normal valency is not exceeded and that the substitution results in a stable compound. If a bond to a substituent is shown to cross the bond connecting two atoms in a ring, then such substituent may be bonded to any atom on the ring. When a substituent is listed without indicating the atom via which such substituent is bonded to the rest of the compound, then such substituent may be bonded via any atom in such substituent. For example, when the substituent is piperazinyl, piperidinyl, or tetrazolyl, unless specified otherwise, such piperazinyl, piperidinyl, or tetrazolyl group may be bonded to the rest of the compound of the invention via any atom in such piperazinyl, piperidinyl, or tetrazolyl group. Generally, when any substituent or group occurs more than one time in any constituent or compound, its definition on each occurrence is independent of its definition at every other occurrence. Such combinations of substituents and/or variables, however, are permissible only if such combinations result in stable compounds.

In a specific embodiment, the term “about” or “approximately” means within 20%, preferably within 10%, and more preferably within 5% of a given value or range.

The yield of each of the reactions described herein is expressed as a percentage of the theoretical yield.

### Experimental Examples

The invention provides processes for making compounds of Formula (X). In all schemes, unless specified otherwise, R¹ to R³ in the formulas below have the meanings of R¹ to R³ in the Summary of the Invention section. Intermediates used in the preparation of compounds of the invention are either commercially available or readily prepared by methods known to those skilled in the art.

The synthesis of a compound of Formula (X) is carried out as shown in Scheme I below.

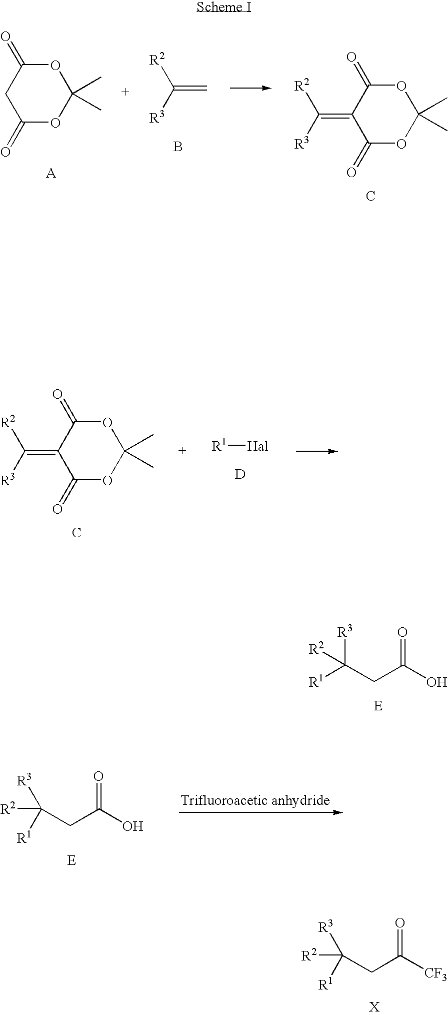

As illustrated in Scheme I, reacting Meldrum's acid of Formula (A) with a carbonyl compound of Formula (B) bearing R² and R³, in a suitable solvent, in the presence of a suitable base, provides the alkylidene-dione of Formula (C). Reacting the alkylidene-dione of Formula (C) with a suitable organohalide of Formula (D) wherein Hal is Br or I, in the presence of a suitable organometallic reagent such as an alkyl magnesium chloride, in a suitable solvent, followed by addition of water and a suitable acid and heating, forms the acid of Formula (E). Reacting the acid of Formula (E) with a trifluoromethyl reagent such as trifluoroacetic acid, in a suitable solvent, in the presence of a suitable base, provides a compound of Formula (X). The compound of Formula (X) may be converted to another compound of Formula (X) by reactions known to one skilled in the art.

Optimum reaction conditions and reaction times may vary depending on the particular reactants used. Unless otherwise specified, solvents, temperatures, pressures, and other reaction conditions may be readily selected by one of ordinary skill in the art. Furthermore, if the substituent groups on R¹ to R³ are incompatible under the reaction conditions of the process, protection/deprotection of these groups may be carried out, as required, using reagents and conditions readily selected by one of ordinary skill in the art, see, for example, T. W. Greene and P. G. M. Wuts, Protective Groups in Organic Synthesis, New York: John Wiley & Sons (1999) and references cited therein. For example, a hydroxyl group can be protected as methyl ether and be deprotected at an appropriate stage with reagents, such as boron tribromide in dichloromethane. Specific procedures are provided in the Experimental Examples section. Typically, reaction progress may be monitored by high performance liquid chromatography (HPLC) or thin layer chromatography (TLC), if desired, and intermediates and products may be purified by chromatography on silica gel by recrystallization and/or distillation.

### Synthetic Example

The following is a representative example that illustrates the process of the invention. HPLC used to determine diastereoselectivity were done on a Supelco SUPELCOSIL™ ABZ+Plus column (4.6 mm×10 cm) eluting with a gradient of 5% acetonitrile/95% water/0.05% TFA to 100% acetonitrile/0.05% TFA over 15 minutes and then held at 100% acetonitrile/0.05% TFA for 5 minutes. References to concentration or evaporation of solutions refer to concentration on a rotary evaporator.

### Example

### Synthesis of 5-Fluoro-N-[(S)-1-(4-methoxyphenyl)ethyl]-2-(4,4,4-trifluoro-1,1-dimethyl-3-oxobutyl)benzamide

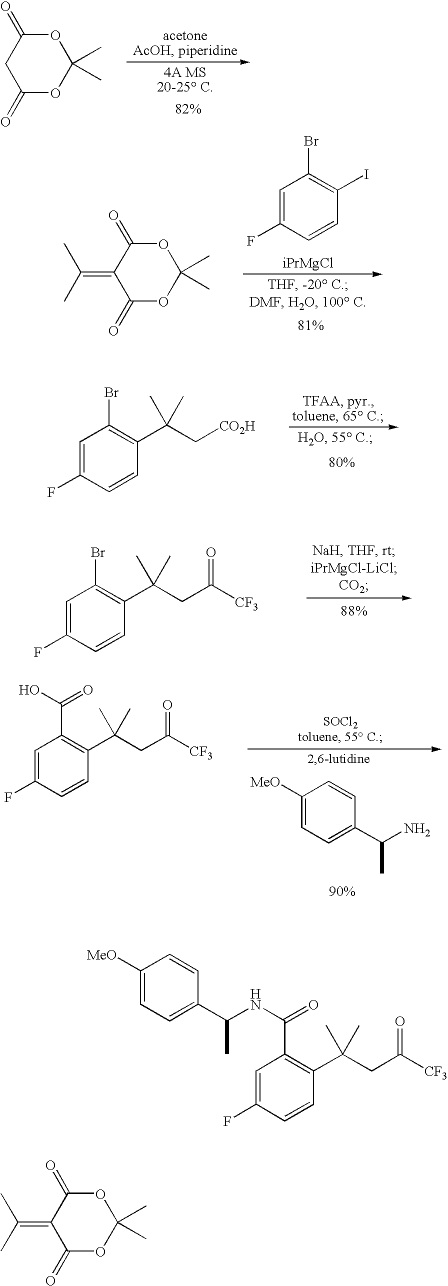

Add 2,2-dimethyl-1,3-dioxane-4,6-dione (Meldrum's acid) (250.0 g) to a dry, nitrogen flushed reactor. Add powdered 4 Å molecular sieves (375.0 g) to the reactor. Add acetone (1.250 L, water content≦0.2%) to the reactor and agitate the slurry for about 10 minutes at 20° C.-25° C. Add acetic acid (0.50 mL) followed by piperidine (2.50 mL) to the reactor and agitate the slurry at 20° C.-25° C. for about 18 hours. Filter the batch to remove the powdered 4 Å molecular sieves. Wash the cake of sieves with toluene (250 mL). Distill the filtrate at 30° C.-35° C. and 100-150 mmHg to remove the acetone. At 35° C., add heptane (1.0 L) to the batch, maintaining the internal temperature at about 35° C. Solid precipitates out during the addition. Decrease the internal temperature to 5° C.-10° C. and hold at this temperature for at least 30 minutes. A thick slurry is obtained. Filter the slurry. Wash the cake with heptane (500 mL) and dry the solid at 20° C.-25° C. and 25-50 mmHg 5-Isopropylidene-2,2-dimethyl-1,3-dioxinane-4,6-dione is obtained as a slightly yellow-white solid, 264.4 g, 82.8% yield.

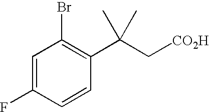

Add 2-bromo-4-fluoro-1-iodobenzene (42.2 mL) and THF (150 mL) to the reactor. Cool the batch to −30° C. and add isopropyl magnesium chloride (162.9 mL, 2.0M/THF) at a rate to maintain the temperature between −30° C. to −20° C. Stir the reaction mixture at about −25° C. to −20° C. for 30 minutes. Add a solution of 5-isopropylidene-2,2-dimethyl-1,3-dioxinane-4,6-dione (50.0 g) in THF (75.0 mL) at a rate to maintain the temperature between −20° C. to −10° C. Stir the batch at −15° C. to −10° C. for 2 hours. Quench the reaction mixture with a solution of concentrated HCl (35.0 mL) in water (120 mL). Add DMF (150 mL), and distill out the THF, residual 3-bromofluorobenzene and other volatiles under vacuum (100-150 mmHg) at 75° C. Heat the batch at 100° C. for 16 to 20 hours. Cool the batch to 20° C.-25° C. and charge a solution of concentrated HCl (25 mL) in water (175 mL). Cool the batch to 0° C.-5° C. and hold it at this temperature for 2 hours. Filter the solid, wash the cake with water, and dry the solid at 55° C.±5° C. under vacuum (−100 mmHg). 3-(2-Bromo-4-fluorophenyl)-3-methylbutyric acid is obtained as a tan solid, 60.5 g, 81.0% yield, 99.4 area % purity by HPLC (220 nm), water content=0.10%.

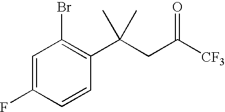

Add 3-(2-bromo-4-fluorophenyl)-3-methylbutyric acid (100.0 g) and toluene (400 mL) to the reactor. Add trifluoroacetic anhydride (TFAA) (151.6 mL) at 25° C. Cool the batch to 0° C.-5° C. Add pyridine (132.3 mL) at a rate that the temperature does not exceed 35° C. Heat the batch to 60° C.-65° C. and hold at this temperature for 12 to 16 hours. Cool the batch to 0° C.-5° C. and quench with water (400 mL) at a rate that the temperature does not exceed 50° C. Heat the batch at 55° C. for 1 to 2 hours. Cool the batch to 20° C.-25° C. Dilute the reaction mixture with heptane (400 mL), agitate it for 5 minutes, allow the layers to settle for 10 minutes, and separate the layers. Treat the batch with water (400 mL), agitate it for 5 minutes, allow the layers to settle for 10 minutes, and separate the layers. Distill the organic phase to the minimum stirrable volume under vacuum (˜150 mmHg) at 60° C.-70° C. Add heptane (600 mL). Filter the dark product solution through a silica gel pad (100 g of SiO₂). Rinse the pad with heptane (600 mL). Distill the light yellow filtrate under vacuum (˜150 mmHg) at 60° C.-70° C. to the minimum stirrable volume. A concentrated solution of 1,1,1-trifluoro-4-(2-bromo-4-fluorophenyl)-4-methyl-2-pentanone (itself an oil in pure form) in heptane/toluene is obtained, 125.0 g, 76.6 wt. % by assay, 80.5% yield.

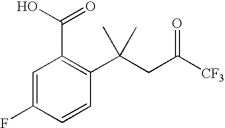

Add sodium hydride (8.80 g, 60 wt. % dispersion in mineral oil) to the reactor under a nitrogen atmosphere. Add THF (150.0 mL) containing 300 to 500 ppm water to the reactor. Cool the slurry to internal temperature of 0° C.-5° C. Add a solution of 1,1,1-trifluoro-4-(2-bromo-4-fluorophenyl)-4-methyl-2-pentanone (109.0 g, 55.0 wt. %) in THF (70.0 mL) at a rate that internal temperature does not exceed 10° C. Heat the batch to 20° C.-25° C. over 30 minutes and set aside the batch at 20° C.-25° C. for 18 hours. Cool the batch to 0° C.-5° C. Add isopropylmagnesium chloride lithium chloride complex (162.12 mL, 1.30 M in THF) at a rate that internal temperature does not exceed 20° C. Add 1,4-dioxane (40.0 mL). Raise the internal temperature to 20° C.-25° C. Set aside the reaction mixture at 20° C.-25° C. for 2 to 3 hours. Cool the batch to internal temperature of −15° C. to −10° C. Bubble carbon dioxide into the reaction mixture at a rate that internal temperature does not exceed 20° C. The carbon dioxide is bubbled in until at least 1.5 equivalents have been added as determined by weight. Set aside the batch at 5° C.-15° C. for 30 minutes and then cool the batch to 0° C.±5° C. Slowly add a solution of concentrated HCl (62.5 mL) in water (187.5 mL) at a rate to control the evolution of hydrogen gas and such that internal temperature does not exceed 30° C. Distill out the THF and isopropyl bromide at batch temperature of not more than 35° C. and 50-100 mmHg Add water (150 mL) to the batch and decrease the temperature to 0° C.-5° C. Hold the batch at 0° C.-5° C. for 2 hours. Filter the solid. Wash the cake with water (200 mL). Dry the solid under vacuum (25-100 mmHg) at 20° C.-25° C. for 8 to 12 hours. This gives 54.7 g of 1,1,1-trifluoro-4-(2-carboxy-4-fluorophenyl)-4-methyl-2-pentanone in 86.1 wt. % purity by assay (88% yield) and 97.2 area % purity by HPLC (220 nm) and with water content of 0.37%.

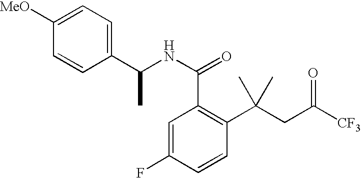

Add 1,1,1-trifluoro-4-(2-carboxy-4-fluorophenyl)-4-methyl-2-pentanone (54.7 g, 86.1 wt. %) to the reactor. Add toluene (250 mL) to the reactor and agitate the slurry at ˜150 rpm. Add thionyl chloride (12.93 mL) followed by dimethylacetamide (0.10 mL). Heat the slurry at an internal temperature of about 55° C.±5° C. for at least 3 hours. On reaching 55° C., the slurry gradually becomes a solution. In a separate reactor add S-1-(4-methoxyphenyl)ethylamine (26.18 mL), 2,6-lutidine (37.1 mL), and THF (100.0 mL). Cool the solution to 0° C.-5° C. Charge the toluene/acid chloride solution to the amine/2,6-lutidine/THF solution at a rate that internal temperature does not exceed 15° C. Set aside the batch at 20° C.-25° C. for 30 minutes. Cool the batch to 0° C.-5° C. Add a solution of concentrated HCl (50.0 mL) in water (200.0 mL) at a rate that internal temperature does not exceed 30° C. and then agitate the batch for 10 minutes. Allow the layers to settle for 10 minutes, and drain the lower aqueous phase. Add water (200.0 mL). Agitate the batch for 10 minutes. Allow the layers to settle for 10 minutes, and drain the lower aqueous phase. Distill the organic phase to the minimum stirrable volume (˜100 mL for this batch) at jacket temperature of 50° C.-65° C. and ˜100-150 mmHg Add heptane (300.0 mL) at a rate to maintain the batch temperature at 65° C.-75° C. Add water (50.0 mL) and hold the temperature at 70° C.-75° C. for 15-30 minutes. Increase the internal temperature linearly from 70° C.-75° C. to about 5° C. over 2 hours. Set aside the batch at about 5° C. for 2 hours. Filter the solid. Wash the solid with heptane (100.0 mL). Dry the solid under vacuum (25-50 mmHg) with a nitrogen bleed at 55° C.±5° C. for 12 hours. This provides 5-fluoro-N—[(S)-1-(4-methoxyphenyl)ethyl]-2-(4,4,4-trifluoro-1,1-dimethyl-3-oxobutyl)benzamide in 90% yield (61.7 g) and 99.1 area % purity by HPLC (220 nm) and with water content=0.10%.

What is claimed is:

1. A process for synthesis of a compound of Formula (X) 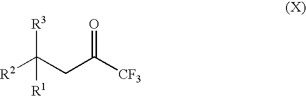
wherein:
R¹ is an aryl group substituted with one to three substituent groups,
wherein each substituent group of R¹ is independently C₁-C₅ alkyl, aminocarbonyl, alkylaminocarbonyl, dialkylaminocarbonyl, halogen, carboxy, cyano, or trifluoromethyl,
wherein each substituent group of R¹ is optionally independently substituted with one to three substituents selected from C₁-C₃ alkyl, C₁-C₃ alkoxy, phenyl, and alkoxyphenyl; and
R² and R³ are each independently C₁-C₅ alkyl,
the process comprising:
(a) reacting Meldrum's acid of Formula (A) with a carbonyl compound of Formula (B) bearing R² and R³, in a suitable solvent, in the presence of a suitable base, to provide the alkylidene-dione of Formula (C) 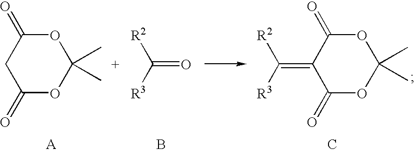
(b) reacting the alkylidene-dione of Formula (C) with a suitable organohalide of Formula (D) wherein Hal is Br or I, in the presence of a suitable organometallic reagent such as an alkyl magnesium chloride, in a suitable solvent, and subsequently adding water and a suitable acid, such as hydrochloric acid, to the reaction mixture and heating to form the acid of Formula (E) 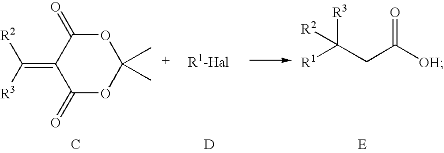
and
(c) reacting the acid of Formula (E) with a trifluoromethyl reagent such as trifluoroacetic anhydride, in a suitable solvent, in the presence of a suitable base, to provide a compound of Formula (X)

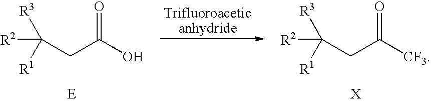

2. The process according to claim 1, wherein:
R¹ is an aryl group substituted with one to three substituent groups,
wherein each substituent group of R¹ is independently C₁-C₅ alkyl, aminocarbonyl, alkylaminocarbonyl, halogen, carboxy, cyano, or trifluoromethyl,
wherein each substituent group of R¹ is optionally independently substituted with one to three substituents selected from C₁-C₃ alkyl, phenyl, and alkoxyphenyl; and
R² and R³ are each independently C₁-C₃ alkyl.

3. The process according to claim 1, wherein the suitable solvent of step (a) is diethyl ether, dipropyl ether, diisopropyl ether, dibutyl ether, THF, DME, MTBE, or a mixture thereof.

4. The process according to claim 1, wherein the suitable base for step (a) is pyridine, piperidine, pyrrolidine, ammonia, or morpholine.

5. The process according to claim 1, wherein the carbonyl compound (B) for step (a) is acetone, cyclohexanone, 2-butanone, 3-pentanone, cyclopentanone, or any other cyclic or acyclic dialkyl ketone.

6. The process according to claim 1, wherein the suitable solvent of step (b) is diethyl ether, dipropyl ether, diisopropyl ether, dibutyl ether, THF, DME, MTBE, toluene, or a mixture thereof.

7. The process according to claim 1, wherein the suitable organometallic reagent of step (b) is isopropylmagnesium chloride, cyclopentylmagnesium chloride, n-butylmagnesium chloride, or tert-butylmagnesium chloride.

8. The process according to claim 7, wherein the suitable organometallic reagent of step (b) is isopropylmagnesium chloride.

9. The process according to claim 1, wherein the suitable solvent of step (c) is dipropyl ether, diisopropyl ether, dibutyl ether, MTBE, toluene, dichloromethane, or a mixture thereof.

10. The process according to claim 1, wherein the suitable base of step (c) is pyridine, 2-chloropyridine, 2,6-lutidine, or 2,4,6-collidine.

11. The process according to claim 1, wherein the suitable trifluoromethyl reagent of step (c) is trifluoroacetic anhydride or trifluoroacetyl chloride.

12. The process according to claim 1, wherein R¹ is an optionally substituted bromophenyl group, further comprising reaction of a compound of Formula (X) with carbon dioxide, in a suitable solvent in the presence of a suitable base and a suitable organometallic reagent, to provide a compound of Formula (X′)

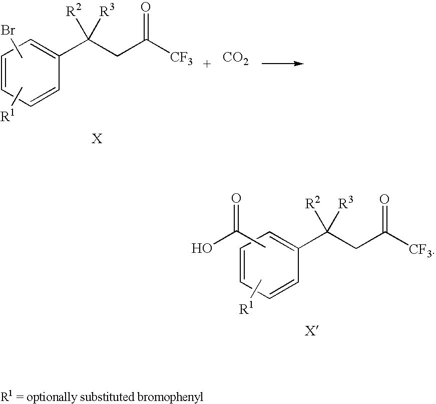

13. The process according to claim 12, wherein the suitable solvent for the reaction of a compound of Formula (X) with carbon dioxide is THF, 2-methyltetrahydrofuran, MTBE, 1,2-dimethoxyethane, or toluene, or a mixture thereof.

14. The process according to claim 12, wherein the suitable base for the reaction of a compound of Formula (X) with carbon dioxide is sodium hydride, lithium hydride, or calcium hydride.

15. The process according to claim 12, wherein the suitable organometallic reagent for the reaction of a compound of Formula (X) with carbon dioxide is isopropylmagnesium chloride, isopropylmagnesium chloride-lithium chloride, n-butylmagnesium chloride, di-n-butylmagnesium, cyclohexylmagnesium chloride, cyclopentylmagnesium chloride, or any other secondary alkylmagnesium chloride.

16. The process according to claim 1, further comprising reaction of a compound of Formula (X′) with a suitable amine, in the presence of suitable reagent, in the presence of a suitable base, in a suitable solvent, to provide a compound of Formula (X″)

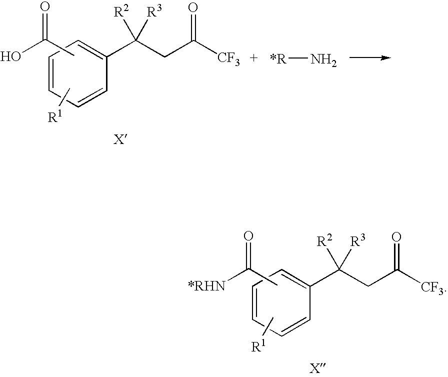

17. The process according to claim 16, wherein the suitable solvent for the reaction of a compound of Formula (X′) with a suitable amine is THF, 2-methyltetrahydrofuran, MTBE, 1,2-dimethoxyethane, or toluene, or a mixture thereof.

18. The process according to claim 16, wherein the suitable amine is any chiral 1-phenylethylamine or any chiral 1-alkyl-1-arylamine.

19. The process according to claim 16, wherein the suitable reagent for the reaction of a compound of Formula (X′) with a suitable amine is thionyl chloride, oxalyl chloride, 1,1′-carbonyldiimidazole, trimethylacetyl chloride, or isobutylchloroformate.

20. The process according to claim 16, wherein the suitable base for the reaction of a compound of Formula (X′) with a suitable amine is 2,6-lutidine, pyridine, 2,4,6-collidine, or ethyldiisopropylamine.
